(19) United States
(12) Patent Application Publication
(10) Pub. No.: US 2007/0155693 A1
(43) Pub. Date: Jul. 5, 2007

(54) Inhibitor of cancer bone metastasis

(75) Inventor: Akikuni Yagita, Tokyo (JP)

Correspondence Address: JHK LAW, P.O. BOX 1078, LA CANADA, CA, 91012-1078, US

(73) Assignee: ORIENT CANCER THERAPY CO., LTD., Tokyo (JP)

(21) Appl. No.: 10/597,202

(22) PCT Filed: Jan. 18, 2005
(86) PCT No.: PCT/JP05/00527

§ 371 Date: Jul. 20, 2006

(30) Foreign Application Priority Data
Jan. 19, 2004 (JP) 2004-011024

(51) Int. Cl. A61K 31/716; A61K 31/663; A61K 31/4152; A61K 31/365

(52) U.S. Cl. 514/54; 514/102; 514/406; 514/471

(57) ABSTRACT

The subject of the present invention is to provide means to fully achieve the inhibition of cancer bone metastasis, which was accomplished through the repeated selection of agents with aiming at obtaining more beneficial effects on the inhibition of cancer bone metastasis. The invention is achieved by combining an inhibition substance of the activation of osteoclast caused by the degradation of a signaling molecule, TRAF6, in the activation of osteoclast, a suppressive substance of the differentiation from osteoclast precursor cells to mature osteoclasts, and/or a bone resorption inhibitor and/or a Cox2 synthesis inhibitor. This combination was found to have an extremely high utility for the inhibition of cancer bone metastasis. Further, the invention is achieved by the inhibitor of cancer bone metastasis, wherein an IL-12 production inducer as an inhibition substance of the activation of osteoclast caused by the degradation of a signaling molecule, TRAF6, in the activation of osteoclast, a tyrosine kinase inhibitor as a suppressive substance of the differentiation from osteoclast precursor cells to mature osteoclasts, and/or a bisphosphonate as a bone resorption inhibitor and/or a Cox2 synthesis inhibitor for inhibiting the stimulation of RANKL/RANK receptor are combined.

### TECHNICAL FIELD

The present invention is to provide a new region of cancer therapy. In particular, an inhibitor of cancer bone metastasis for a novel method for preventing and treating cancer bone metastases is provided.

This application claims the priority of Japanese Patent Application No. 2004-011024, which is incorporated herein by reference.

### BACKGROUND ART

Bone is a favorite organ for metastasis of cancer. Growth factors such as TGFβ and IGFs are stored abundantly in bone. These growth factors are released at the time when osteoclasts absorb bone, thereby cancer cells beginning to colonize in bone are assured their proliferation and metabolism. Stimulated cancer cells produce cytokines, growth factors, which activate osteoclasts or osteoblasts, to form and augment osteoclastic or osteogenic bone metastasis. Like this, bone metastasis may be regarded as a lesion formed by a collaboration work between cancer cells and bone. Breaking this relation leads to an approach for effective inhibition of bone metastasis (non-patent document No. 1). There are two types of osteopathy associated with malignant tumor. The one is a case that cancer cells directly metastasize to bone, promoting the formation of osteoclasts at the metastasis site to activate and proliferate the osteoclasts. The other is a case that though cancer cells don't directly metastasize to bone, PTH-rP (Parathyroid Hormone Related Protein) which is produced from cancer cells proliferate and activate osteoclasts. In either of the pathologies, formation, proliferation, activation and also life extension of osteoclasts lead to weakening bone tissues in a whole body, resulting in osteopathy involving pain or fracture. The bone metastasis is accompanied by not only severe pain but dyskinesia. In addition, when fractured, the symptom becomes more intense. Further, it has been known that the onset of hypercalcemia resulted from bone metastasis may directly be life-threatening.

At present, as a therapy for this bone metastasis, surgery, radiotherapy, anticancer agents, hormone therapy and the like may be mentioned, but each of the therapies is temporary or local, while extremely limited. Further, when multiple bone metastasis is combined, nothing can do for it. Disorders often involving bone metastasis or osteopathy are multiple myeloma; breast, prostate, head and neck, lung, renal and ovarian cancers; malignant lymphoma, gastric cancer and the like.

Previously, as an innovative approach in cancer therapy, the present inventor Yagita, M. D., focused on the utility of a substance which induces interleukin 12 (IL-12) in vivo, discovered a processed mycelium of mushroom had that function, and established a cancer therapy so called Novel Immunotherapy for Cancer (NITC). There is a fact that conventional IL-12 has anticancer effects but when directly administered in vivo, it provides side effects, making the therapy unacceptable for patients, thus IL-12 itself could not be used as an anticancer agent. However, formulations comprising the processed mycelium of mushroom reported by Yagita achieved remarkable therapeutic and life-prolonging effects in cancer therapy. Namely, Yagita achieved the goal of cancer therapy by administering an effective amount of a processed mycelium of mushroom enough for inducing IL-12 in vivo. (patent document No. 1).

IL-12 has effects of activating and enhancing killer T cells by the route of TNFα→IFNγ→IL-12 CTL activities. Therefore, anticancer effects are expected by increasing IL-12 production through activation and enhancement of killer T cells.

Molecular-targeting therapeutic agents for cancer have been focused on their meaning as a new type antitumor drug in contrast to conventional cellular-targeting therapeutic agents. Among them, in particular, tyrosine kinase inhibitor attracts the attention as an agent having an inhibitory effect of signal transduction. ZD1839 (Iressa® of AstraZeneca K.K.) shows a competitive effect for ATP in ATP binding site of EGFR (epidermal growth factor receptor) tyrosine kinase, and inhibits tyrosine kinase activity by inhibiting autophosphorylation of tyrosine kinase. As a result, the anticancer effect is expressed by blocking an EGFR-equipping signal transduction {ligands such as epidermal growth factor (EGF) are bound to the extracellular domain of EGFR, followed by activation of EGFR tyrosine kinase in the intracellular domain, causing not only autophosphorylation of EGFR but also phosphorylation of various intracellular target proteins, then transducing a proliferation signal from the cell surface to nucleus, then transducing the proliferation signals from the cancer cell surface to nucleus, and resulting in proliferation, infiltration, metastasis, angiogenesis of cancer cells} in association with proliferation, infiltration, differentiation and metastasis. IMC-C225 (EGFR-targeting monoclonal antibody) recognizes the receptor part of EGFR on a cell membrane surface and inhibits the autophosphorylation of EGFR thereby inhibiting the tyrosine kinase activity. Herceptin is a monoclonal antibody against Her2/Neu which is homologous to EGFR, and STI-571 (Gleevec) can inhibit both tyrosine kinase activities of BCR-Abl and c-kit (non-patent document No. 2).

Such molecular-targeting therapeutic agents have attracted attentions as cancer therapeutic drugs by a new mechanism, but their effects still cannot be called revolutionary. For example, ZD1839 (Iressa) is a potent and selective EGFR tyrosine kinase inhibitor newly developed by AstraZeneca K.K. and found useful in human. However, against non-small cell lung cancer and prostate cancer, its clinical data showed PR (partial remission) of 10 to 20-something % and CR (complete remission) of almost nothing, and if any, the cases were quite rare and took a period of more than four months until complete remission. Accordingly, therapies combining ZA1839 (Iressa) and various anticancer agents have been attempted, but as of now, additive or synergistic effects have not been obtained.
Patent document No. 1: Japanese Patent Application Laid-open No. Hei 10-139670
Non-patent document No. 1: “Experimental Medicine” Vol. 20, No. 17 (extra) 2002
Nonpatent document No. 2: “Blood-Immunity-Cancer” Vol. 7, No. 3, 2002-7

### DISCLOSURE OF THE INVENTION

### Problem to be Solved by the Invention

The present invention aims at exerting more beneficial effects on the inhibition of cancer bone metastasis, and through repeatedly selecting agents and heightening that inhibition rates, provides a complete inhibition means of cancer bone metastasis.

### Means for Solving the Problem

The present invention found that centering on an inhibition substance of the activation of osteoclast caused by the degradation of a signaling molecule, TRAF6, in the activation of osteoclast, combining a suppressive substance of the differentiation from osteoclast precursor cells to mature osteoclasts, and/or a bone resorption inhibitor and/or a Cox2 synthesis inhibitor for inhibiting the stimulation of RANKL/RANK receptor leads to an extremely high utility for the inhibition of cancer bone metastasis, and completed the present invention.

Therefore, the present invention consists of:
1. A inhibitor of cancer bone metastasis, wherein an inhibition substance of the activation of osteoclast caused by the degradation of a signaling molecule, TRAF6, in the activation of osteoclast, a suppressive substance of the differentiation from osteoclast precursor cells to mature osteoclasts, and/or a bone resorption inhibitor and/or a Cox2 synthesis inhibitor are combined.
2. A inhibitor of cancer bone metastasis, wherein an IL-12 production inducer as an inhibition substance of the activation of osteoclast caused by the degradation of a signaling molecule, TRAF6, in the activation of osteoclast, a tyrosine kinase inhibitor as a suppressive substance of the differentiation from osteoclast precursor cells to mature osteoclasts, and/or a bisphosphonate as a bone resorption inhibitor and/or a Cox2 synthesis inhibitor for inhibiting the stimulation of RANKL/RANK receptor are combined.
3. A inhibitor of cancer bone metastasis, wherein an inhibition substance of the activation of osteoclast caused by the degradation of a signaling molecule, TRAF6, in the activation of osteoclast, a suppressive substance of the differentiation from osteoclast precursor cells to mature osteoclasts, and/or a bone resorption inhibitor and/or a substance enhancing the production of osteoprotegerin are combined.
4. The inhibitor of cancer bone metastasis according to any one of preceding items 1 to 3, wherein the tyrosine kinase inhibitor has a selectively targeting effect to at least one receptor from the followings:
HER2/neu, HER3, HER4, c-kit, PDGFR, bcr-ab1 and EGFR.
5. The inhibitor of cancer bone metastasis according to any one of preceding items 1 to 4, wherein IL-12 production inducer is a substance having a β1,3/1,6 glucan structure.
6. A method for preventing and treating cancer bone metastases by the inhibitor of cancer bone metastasis according to any one of preceding items 1 to 5.

### Effects of the Invention

In the present invention, centering on an inhibition substance of the activation of osteoclast caused by the degradation of a signaling molecule, TRAF6, in the activation of osteoclast, and by combining a suppressive substance of the differentiation from osteoclast precursor cells to mature osteoclasts, and/or a bone resorption inhibitor and/or a Cox2 synthesis inhibitor, clinical data showing an extremely high inhibition of cancer bone metastasis were achieved. In the present invention, the inhibition of differentiation and induction of osteoclast are based on blocking an intracellular transduction system which comes from TNFα, RANKL, IL-1 and the like, and the specific methods are divided into four (FIG. 6).

### BRIEF DESCRIPTION OF THE DRAWINGS

FIG. 1 shows a scheme of formation of an osteoclast.

FIG. 2 shows a scheme of NITC therapy.

FIG. 3 shows a scheme of inhibiting the formation of osteoclast for treating osteopathy.

FIG. 4 shows clinical cases. (Example 1)

FIG. 5 shows clinical cases. (Example 2)

FIG. 6 shows a scheme of the therapy of present invention.

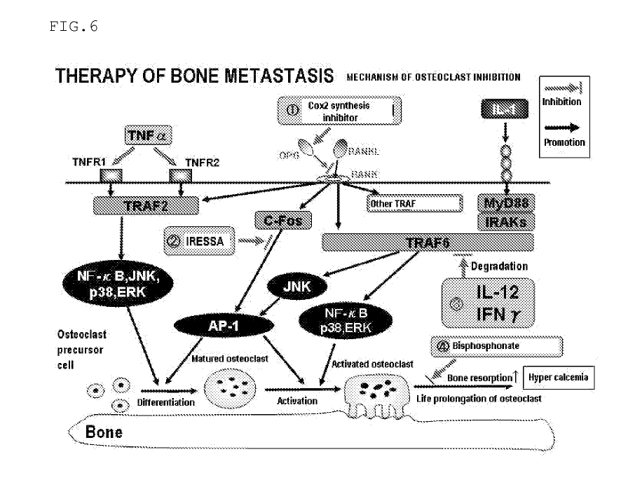

### DESCRIPTION OF THE PREFERRED EMBODIMENT

The present invention will be explained in detail below and technical and scientific terms used herein have, unless specified otherwise, meanings usually understood by those ordinarily skilled in the art to which the present invention belongs.

Novel Immunotherapy for Cancer (NITC) by the present inventor Yagita, M. D., is a therapeutic means formed by combining four different mechanisms of action. The first mechanism of action is the method of administering an angiogenesis inhibitory substance (better shark) to interfere with blood flow into cancer, thereby reducing the cancer. The effect of this can be determined by measuring vascular endothelial growth factor (VEGF). Angiogenesis inhibitory effects can be evaluated by minus (negative) value of VEGF (−VEGF). The angiogenesis inhibitory capacity can also be evaluated using the other vascular growth factors such as FGF and HGF instead of this VEGF value. In addition, the evaluation can also be conducted with positive values (for example, endostatin value) of the angiogenesis inhibitory factor instead of VEGF.

The second mechanism of action is the method of activating CTLs by administering a compound having a β1,3-glucan structure, thereby inducing Th1 cytokine (TNFα, IFNγ and IL-12). While CTL activity can be determined by the capacity of CD8(+) to produce perforin, there are two types of in this CD8(+) perforin value, cytotoxic T cell (CTL) and immunosuppressive T cell (STC, Suppressor T cell), and the former impairs cancer cells, and the activation of the latter results in the proliferation of cancer. Therefore, the absolute value of it cannot provide the evaluation. However, if IFNγ is 10 IU/ml or more, or IL-12 value is 7.8 pg/ml or more, the former should be CTL, and if both IFNγ and IL-12 show lower values, it should be determined STC. Thus, CTL activities can be evaluated by the capacity to produce either IFNγ (IFNγ value) or IL-12 (IL-12 value).

Effector cells activated by the administration of a compound having α1,3-glucan structure, which is the third and fourth mechanisms of action, are NK and NKT cells. These NK and NKT cells share NKR-P1 (NK cell receptor CD161(+)), and the cell number of the former NK cell can be countered by the surface markers of CD3(−)CD161(+) and its activation can be determined by the capacities of CD3(−)CD161(+) to produce perforin. Whereas, the cell number of the latter NKT cell can be countered by the surface markers of CD3(+)CD161(+)and its activation can be determined by the capacities of CD3(+)CD161(+) to produce perforin (referred to as NKTP(+)).

Therefore, even if it is a novel immunotherapy (NITC) or common immunotherapy in cancer therapy, its effector cells or angiogenesis inhibitory effects can be evaluated respectively with the following determination items. In particular, CTL activity can be evaluated by the inducing capacities of producing IFNγ or IL-12. The activation of NK cell can also be evaluated by either CD3(−)CD161(+) or CD3(−)CD161(+) perforin value. The activation of NKT cell can also be evaluated by either CD3(+)CD161(+) or CD3(+)CD161(+) perforin value (NKTP value).

The present invention is provided by combining each inhibitor of tyrosine kinase, and/or bone resorption and/or Cox2 synthesis in addition to an IL-12 production inducer in the novel immunotherapy described above.

As an IL-12 production inducer for use in the present invention, for example, a compositional formulation of mushroom mycelium having a β1,3-glucan structure (for example, ILX™ from Touzai Iyaku Kenkyusho K.K.; ILY™ from Seishin Enterprise Co., Ltd.), or various yeasts having a β1,3-glucan structure (marine yeast, bread yeast, NBG™) can be used. In particular, marine yeasts are preferred. The IL-12 production inducer for use in the present invention has an inhibitory effect on the activation of osteoclast caused by the degradation of a signaling molecule, TRAF6, in the activation of osteoclast, and substances having such a function are broadly applicable. The IL-12 production inducer for use in the present invention will be used in a prescription capable of inducing or enhancing its production inducer activity, and further maintaining its activity. Therefore, it is used by selecting both dosage and administration period which will induce or enhance its activity, and further maintain that activity. In particular, for the dosage, a CTL activator (IL-12 and INFγ production inducers), which is a compound having a β-1,3 glucan structure, will be administered at approximately 1 g to 10 g/day, preferably approximately 3 g to 6 g/day. Further, the treatment period will be usually for 10 days to 24 months, and the dosages be from alternate day or one to three times/day, and daily administration is preferred. The CTL activator (IL-12 and INFγ production inducers) of interest will preferably be orally administered.

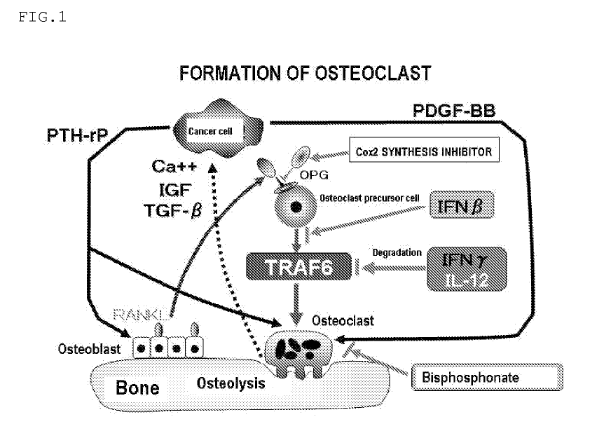

The relevance between IL-12 production inducer and the degradation of a signaling molecule, TRAF6, in the activation of osteoclast will be explained. Osteopathy in a malignant tumor patient is caused in such a manner that an osteoclast precursor cell adheres to a stimulated RANK receptor by a RANKL (a kind of cytokine), leading to the proliferation of TRAF6 in the osteoclast, and finally through either a signal transduction system of NF-κB or AP-1 formed from a JNK gene, the formation of a mature osteoclast is established, and then proliferation and activation are enhanced. When bone tissues are broken, these osteoclasts release TGFβ, IGF and Ca⁺⁺ which are present rich in the bone tissues. These substances present in bone tissues have the effects to proliferate and activate cancer cells. In addition, as cancer cells proliferate, they boost the high production of PTH-rP which has an effect of stimulating osteoblasts and osteoclasts to activate. In summary, a vicious cycle is established wherein the facilitation of osteoclasis proliferates cancer cells, which further facilitates the osteoclasis. Therefore, a significance lies in breaking the vicious cycle of osteopathy by using NITC, in particular, CTL activator (IL-12 and INFγ production inducers) to induce Th1 cytokine, for example, IFNγ and IL-12, thereby degrading TRAF6 of osteoclast (FIG. 1)

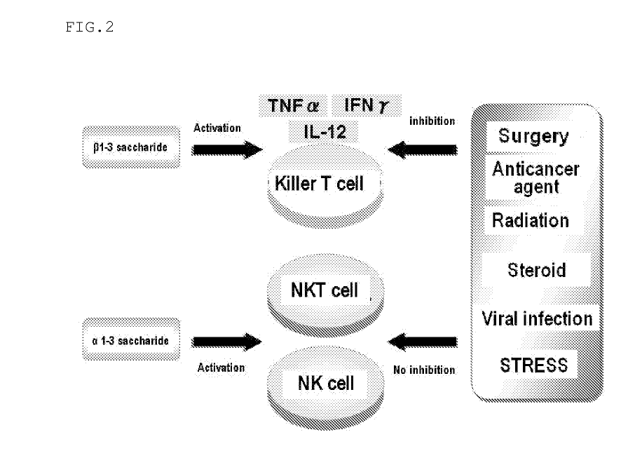

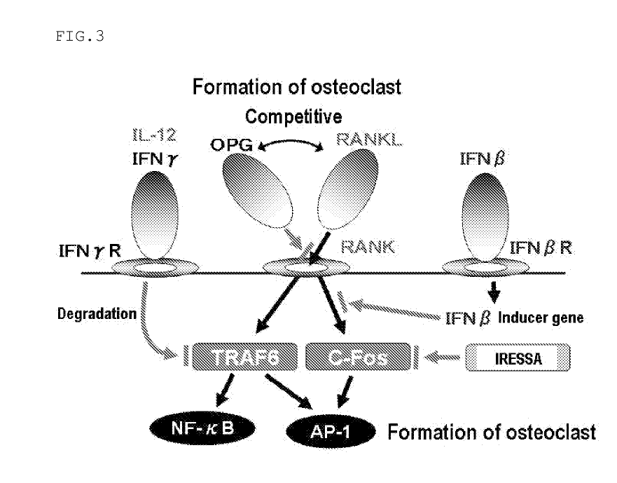

The therapy is such that NITC, in particular, CTL activator (IL-12 and INFγ production inducers) induces intrinsic Th1 cytokines (IFNγ and IL-12) to activate not only CTLs but also NK and NKT cells and the like, thereby enhancing antitumor effects (FIG. 2). These Th1 cytokines degrade TRAF6 which is important in the formation of osteoclasts. As a result, NITC, in particular, CTL activator (IL-12 and INFγ production inducers) inhibits the formation of osteoclasts, thereby treating osteopathy (FIG. 3). Meanwhile, any of many surgeries, radiations and anticancer agents currently conducted in cancer therapies is to inhibit the production of Th1 cytokines, so that they have to be referred to as therapies worsening the bone metastasis.

Therapeutic Effects of NITC Therapy on Bone Metastasis

NITC was conducted in bone metastasis-confirmed 143 cases of breast, lung and prostate cancers, and as a result its effectiveness was confirmed in 82 cases (57.3%). Whereas 55 cases (38.5%) were unchanged and 6 cases (4.2%) ineffective. The criteria herein determined those occasions effective, when among cases wherein bone metastasis had been confirmed positive by a diagnostic imaging such as bone scintigraphy, MRI, CT and the like, 1CTP value of bone metastasis marker decreased by 25% or more compared to the pretreatment. Whereas the cases wherein the change of 1CTP value is within 25% or less compared to the pretreatment were determined unchanged, and the cases with 25% or higher increase ineffective.

1) Therapeutic Effects on Bone Metastasis Cases in Prostate Cancer

Of 65 subjects, 48 cases (73.8%) were effective, 16 cases (24.6%) unchanged and 1 cases (1.5%) ineffective.

2) Therapeutic Effects on Bone Metastasis Cases in Breast Cancer

Example cases of bone metastasis in breast cancer were 50, and 23 cases (46.0%) were effective, 24 cases (48.0%) unchanged and 3 cases (6.0%) ineffective.

3) Therapeutic Effects on Bone Metastasis Cases in Lung Cancer

Example cases of bone metastasis in lung cancer were 28. In determining effect by 1CTP, 11 cases (39.3%) were effective, 15 cases (53.6%) unchanged and 2 cases (7.1%) ineffective.

As a signal transduction system via RANKL, another pathway not mediated by TRAF6, i.e., a pathway of forming osteoclasts through AP-1 formed by a c-Fos gene has been found. NITC exhibited therapeutic outcomes described above, but still satisfaction could not be obtained. In other words, there were cases wherein NITC could not produce an enough amount of IFNγ and IL-12, or cases wherein though an enough amount of Th1 cytokines were produced, the formation of osteoclasts via c-Fos pathway was left. In such a case, a tyrosine kinase inhibitor having inhibitory effects on c-Fos, Iressa, works effective.

In the present invention, the combination of this inhibition substance of the activation of osteoclast, representatively IL-12 production inducer, caused by the degradation of a signaling molecule, TRAF6, in the activation of osteoclast, with tyrosine kinase inhibitor is useful. The tyrosine kinase inhibitor has an inhibitory effect on the differentiation from an osteoclast precursor cell to a mature osteoclast, and a substance having such function can broadly be applied. As a specific example of tyrosine kinase inhibitor, ZD1839 (Iressa, trade name) or STI571 (Gleevec, trade name) may be mentioned and various types of tyrosine kinase inhibitors can be effectively used. As those targeting molecules, HER2/neu, HER3, HER4, c-kit, PDGFR, bcr-ab1, EGFR and the like may be mentioned. The most effective molecule is EGFR or c-kit. The dosage of tyrosine kinase inhibitor will follow the recommended one for each molecular-targeting compound and doses of 10 to 500 mg/day will orally be administered.

The cases of Examples 1 and 2 are those wherein though Th1 cytokines, IFNγ and IL-12, were produced in large amounts, the improvements of bone metastasis and skeletal pain were not observed. In those cases, it was suggested that the formation of osteoclast might be maintained by the pathway through c-Fos, and advantageous results were obtained by the combination of a tyrosine kinase inhibitor therewith.

In the present invention, centering on an inhibition substance of the activation of osteoclast, representatively IL-12 production inducer, caused by the degradation of a signaling molecule, TRAF6, in the activation of osteoclast, combining a suppression substance of the differentiation from osteoclast precursor cells to mature osteoclasts (representatively, tyrosine kinase inhibitor) and/or a bone resorption inhibitor is useful. As a bone resorption inhibitor, bisphosphonate is typical. As a bisphosphonate formulation, for medicines for internal use, there are disodium etidronate 200-1000 mg/day (Didronel®), alendronate sodium hydrate 5 mg/day (Fosamac®, Bonalon®), sodium risedronate hydrate 2.5 mg/day (Benet®, Actonel®) and the like, for injection medicines, there are disodium pamidronate 30-45 mg/day (Aredia®), alendronate sodium hydrate 10-20 mg/day (Onclast®, Teiroc®), disodium incadronate 10 mg/day (Bisphonal®) and the like. Further, though not approved in Japan, for medicines for internal use, there are ibandronate 10-50 mg/day (BONIVA), clodronate 1600-3200 mg/day (Bonefos®, Ostec®), tiludronate 240 mg/day (Skelid®) and the like, and for injection medicines, there are such as zoledronate (Zometa®) and ibandronate 2-4 mg/time.

Further, in the present invention, combining a Cox2 synthesis inhibitor for inhibiting the stimulation of RANKL/RANK receptor therein is also effective. That is because the synthesis of Cox2 inhibits the producion of osteoprotegerin (OPG) which is an inhibitory cytokine to RANKL. Namely, combining a substance enhancing the production of osteoprotegerin, for example, prostaglandin E2 synthase of Cox2 synthesis inhibitor described above therewith is effective. As a Cox2 synthesis inhibitor, there are aryl acetates such as etodolac 400 mg/day (Hypen®, osteluc®, Etopen®, Ospain®, Niconas®, Hisrack®, Hypelac®, Raipeck:®), Oxicams such as Meloxicam 10 mg/day (Mobic®), and though not approved in Japan, there are celecoxib 200 mg/day (Celebrex), rofecoxib 12.5-25 mg/day (Biox), valdecoxib 10-20 mg/day (Bextrah) and Nimesulide.

Selective combination of these four kinds may be conducted from the beginning of treatment, or any one may precede the others. As a specific example, a dramatic clinical effect was observed when a tyrosine kinase inhibitor and/or a bisphosphonate and/or a Cox2 synthesis inhibitor were combined after administering an NITC therapeutic agent, in particular, a cancer immunotherapeutic agent for a certain period.

In the present invention, as a cancer immunotherapeutic agent, besides IL-12 production inducer, NK or NKT activator may be combined. A compositional formulation of a compound having a α1,3-glucan structure such as nigerooligosaccharide and fucoidan is useful as NK activator or NKT active agent. A variety of compounds having a α1,3-glucan structure are known, and combining this previously known structure, with the measurement of CD3(−)CD161(+) and CD3(−)CD161(+) perforin producing capacities, and CD3(+)CD161(+) and CD3(+)CD161(+) perforin producing capacities will allow those skilled in the art to easily specify the NK activator. Now, CD3(+)CD161(+) means effects on receptor NKR-P1 of NKT cells.

By selecting an application method, the combination of the present invention is effective in treating lung cancer (lung squamous cell carcinoma, lung adenocarcinoma, small cell lung cancer), thymoma; thyroid, prostate, renal, bladder, colonic, rectal, esophageal, cecum, urinary duct, breast, cervical, brain, tongue, pharyngeal, nasal passage, laryngeal, gastric, liver cancers; cholangiocarcinoma; testicular, ovarian, endometrial, metastatic bone cancers; malignant melanoma, osteosarcoma, malignant lymphoma, plasma cell tumor, liposarcoma and the like. The bone metastasis of these cancers can be dominantly inhibited.

When the combination therapy of anticancer agents (chemotherapy), radiation, or steroid is conducted in addition to the present combination, within two kinds of immune systems, the line TNFα→IFNγ→IL-12→killer T cell is significantly interfered. Therefore, they will not preferably be used in the present invention. However, when an anticancer agent is administered, the application of an administration method of a low concentration chemotherapy which is an administration method never interfering with immune system described above, i.e. low concentrations of 5FU, UFT, Mifurol, furtulon, and CDDP (5 μg to 10 μg); and low concentration anticancer agents such as Taxotere or Taxol, adriamycin, mitomycin, CPT-11 and the like is useful. Further and likewise, the application of a low volume irradiation in a radiation therapy as well as low concentration administration or the like in a steroid therapy needs to be selected.

Measuring method of cell and each cytokine will be presented below.

(Measurement of NKT Cell) (Measurement of NK Cell) (Measurement of CD8)

The measurement of NKT cell having NKR-P1 can be conducted by measuring cell surface antigens (CD3 and CD161) which are specifically present on the NKT cell surface. In particular, lymphocytes in peripheral blood are tested for cells of CD3 positive and CD161 positive [CD3(+)CD161(+)]. Namely, CD3 and CD161 which are cell surface antigens of NKT cell are measured by Two Color assay which uses monoclonal antibodies and flow cytometry. Here, NKT cell being activated means that the ratio of NKT [CD3(+)CD161(+)] cells in lymphocytes is 10% or more, and more preferably 16% or more. The capacity of activating NKT cell means the function which can increase NKT cell ratio by 10% or more, and more preferably 16% or more, or which can further increase NKT cell ratio more than that before administering a certain substance. Likewise, [CD3(−)CD161(+)] means to assay for CD3 negative and CD161 positive cells. This method is useful in measurement of NK cells. In addition, CD8(+) means to assay for CD8 positive cells. This method is useful in measurement of CTL activity.

In the Examples, bloods from cancer patients were used, and cells in blood were separated into positive and negative for cell surface antigens, CD3, CD161 and CD8, and each cell ratio was measured by Two Color assay using flow cytometry in a conventional manner. At this time, monoclonal antibodies used against CD3, CD161 and CD8 were supplied by respectively Coulter or Becton & Dickinson.

(Measurement of Perforin Producer Cell)

For lymphocytes in peripheral blood, two of cell surface antigens, CD3, CD161 and CD8 and perforin are measured by Three Color assay using flow cytometry in a conventional manner. Specifically, into a collected blood, a fixative is added to fix cells, then after adding a membrane permeable solution, anti-perforin antibody (supplied by Pharmingen) is added for reaction, further PRE-Cy5-labeled secondary antibody (from DAKO) is added for reaction, then anti-CD3-PE (Coulter 6604627) antibody and anti-CD161-FITC (B-D) antibody are added for reaction, thereafter measurement is conducted by flow cytometry. They were abbreviated as P or PER in figures and a table.

(Preparation of Samples to Measure Cytokines)

Firstly, a monocyte fraction is separated from the blood for preparation. After the heparinized peripheral blood is diluted twofold with Phosphate Buffer Saline (PBS) and mixed there, the mixture is overlaid on Ficoll-Conray solution (specific gravity 1.077), and spun down at 400 G for 20 minutes, then the monocyte fraction is collected. After washing it, RPMI-1640 medium added with 10% fetal bovine serum (FBS) is added there and preparation is made such that the cell number becomes 1×10^{6. Into} 200 μl of the obtained cell suspension, Phytohemagglutinin (supplied by DIFCO) is added up to a concentration of 20 μg/ml, then cultured in a 96-well microplate in the presence of 5% CO₂ at 37° C. for 24 hours, and a sample to measure cytokines in the cultured cell solution is obtained.

(Measurement of IL-12)

To measure the amount of IL-12, a well known clinical and biochemical examination can be used, and a measuring kit based on enzyme immunoassay (ELISA) available from R&D SYSTEMS and MBL is used. Herein, measuring kit supplied by R&D SYSTEMS was used. In practice, into each well of a 96-well microplate, 50 μl of measuring diluent of Assay Diluent RD1F, and 200 μl of standard or sample prepared from the preparation method for measuring cytokines described above were dispensed, then allowed to stand and react at a room temperature for 2 hours. Thereafter, 200 μl of horse radish peroxidase (HRP)-labeled anti-IL-12 antibody was dispensed there and allowed to stand for 2 hours at a room temperature. The reaction solution was removed from each well and washed three times, then 200 μl of color development substrate solution was dispensed and allowed to stand for 20 minutes at a room temperature, and then 50 μl of enzyme reaction terminating solution was dispensed. Using 550 nm as control, the absorbance of each well at 450 nm was measured with Emax (supplied by Wako Pure Chemical Industries, Ltd.). The amount of IL-12 is expressed as pg/ml. Herein, the capacity of inducing IL-12 production means the function which can increase the amount of produced IL-12 from the peripheral blood monocyte fraction by stimulation up to 7.8 pg/ml or more, or which can increase the amount of produced IL-12 more than that before administering a certain substance.

(Measurement of IFNγ)

The measurement of IFNγ was conducted by enzyme immunoassay (EIA method) using IFNγ EASIA kit from BioSource Europe S. In practice, into a each well of a 96-well microplate, 50 μl of standard or the twofold dilution of prepared sample described above was dispensed, then 50 μl of HRP-labeled anti-IFNγ antibody was dispensed, and further allowed to react for 2 hours at a room temperature with shaking. The reaction solution was removed from each well and washed three times, then 200 μl of a color development substrate solution was dispensed, allowed to react for 15 minutes at a room temperature with shaking, and then 50 μl of an enzyme reaction terminating solution was dispensed. Using 630 nm as a control, the absorbance of each well at 450 nm and 490 nm was measured with Emax (supplied by Wako Pure Chemical Industries, Ltd.). The amount of IFNγ is expressed as IU/ml.

(Measurement of Capacity of Inhibiting Angiogenesis)

(Measurement of vascular endothelial growth factor/VEGF and basic fibroblast growth factor/bFGF and angiogenesis inhibitory factor endostatin/Endostatin) Using a commercially available kit, the serum concentration was measured by each enzyme immunoassay solid phase method (ELISA; enzyme linked immuno-sorbent assay) (ACCUCYTE Human VEGF, ACCUCYTE Human bFGF, ACCUCYTE Human Endostatin: CYTIMMUNE Sciences Inc.).

(Measurement of Bone Metastasis)

Effects of inhibiting bone metastasis were confirmed by the variation of 1CTP which is a bone metastasis marker. 1CTP has been practically used as a specific marker for bone resorption and there is, for example, 1CTP kit supplied by Orion (Clin. Chem. 39: 635-640, 1993). The kit uses a decalcified human thigh bone, which is degraded and purified in vitro by a purified bacterial collagenase, as an antigen (1CTP), to immunize a rabbit to produce an antibody, and the antigen is labeled with ¹²⁵I to make competitive radioimmunoassay (RIA). Its measuring sensitivity is 0.5 ng/ml or less and the measurement is possible to at least 50 ng/ml. Further, the mean value in blood level of 287 cases from 25-year old to 72-year old is 3.00±1.12 ng/ml (average±S.D.). 1CTP in blood has shown abnormal values in various metabolic bone diseases such as bone metastasis and hyperparathyroidism by now, and further has also been recognized to correlate with therapeutic process. The kit to measure 1CTP is a known clinical diagnosis one, and there is a RIA kit, pyridinoline 1CTP “Chugai” manufactured by Chugai Diagnostics Science Co., Ltd. Specifically, it is a radioimmunoassay reagent using ¹²⁵I as a label. However, other well known reagents such as enzyme immunoassay reagent using an enzyme labeling and fluorescence immunoassay reagent using a fluorescent substance can of course be applied. The measuring technique is explained in detail in a commercial kit, so not mentioned. In a literature, a reference can be found in “Nuclear Medicine 30:1411 to 1417, 1993”. The representative standard curve for measuring 1CTP is also explained in a kit. The lowest detection sensitivity is 0.5 ng/ml or less. Further, a cut-off value in use as a marker for bone metastasis is 4.5 ng/ml.

Meanwhile, any of markers used in the clinical examination were commercial products and the measured values were exhibited in each recommended manner. Abbreviations expressed were based on each general expression way.

Determination of effects on patients adopted the following five-graded determination: CR (complete remission), PR (partial remission), LNC (long-term no change), SNC (short-term no change) and PD (progressive disease state). Further, the response rate in each cancer species expresses the rates of CR, PR, LNC, SNC and PD to all cases combining each cancers species.

### EXAMPLE

The present invention will be specifically explained below using Examples, but the present invention will not to be limited by those Examples.

A therapy has been conducted on progressive terminal cancer cases as novel immunotherapy (NITC). This NITC is BRM therapy wherein the administration of β-1,3 glucan induces intrinsic TNFα, IFNγ and IL-12 to activate CTLs (killer T cell), the administration of α-1,3 glucan activates NK and NKT cells and further the oral administration of better shark inhibits angiogenesis. Patients were dosed with a cancer immunotherapeutic agent, IL-12 production inducer, Shark cartilage (Seishin Enterprise Co., Ltd.), and a saccharide having a α1,3-structure and the like, following each recommended prescription. Further, as an IL-12 production inducer, ILX (Touzai Iyaku), ILY (Seishin Enterprise Co., Ltd.), Krestin (Sankyo), Immutol (NBG) and the like were administered alone or in combination met considering patients' symptoms.

### Example 1

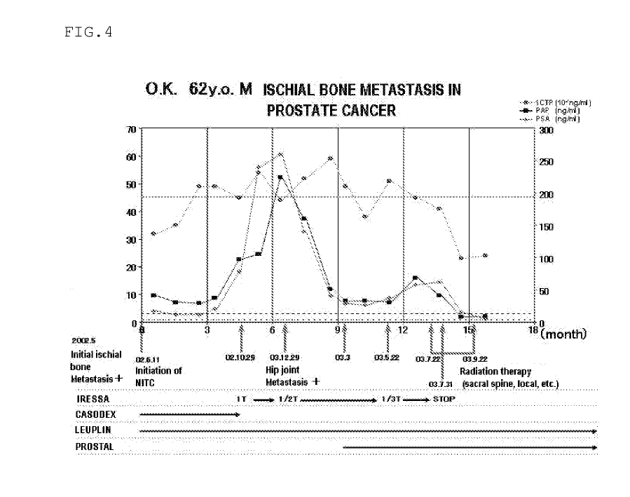

(FIG. 4) 62-year old, a case of prostate cancer and ischial bone metastasis

A prostate cancer patient having been diagnosed as ischial bone metastasis received the hormone therapy of Casodex and Leuplin. NITC was started on Jun. 11, 200X. PSA and PAP of prostate cancer markers were determined NC, but 1CTP which is a bone metastasis marker kept increasing while enough activities of IFNγ and IL-12 were obtained. On Sep. 18, 200X, severe low back pain and left hip joint and hip (ischial bone metastasis) pains were appeared, so morphine (MS Contin 40 mg/day) was used. From Oct. 29, 200X, Iressa 1T (250 mg/day) was started to use. Two months after the administration of Iressa, as the pains in low back, left hip joint and hip were alleviated, the administration of MS Contin decreased to 20 mg/day. Therefore, the dosage of Iressa was changed to 250 mg/alternate day, but PSA and PAP kept decreasing significantly even afterward and the increase of 1CTP also terminated, showing a tendency to decrease. From July, 22 of the next year, radiation therapy to sacral spine was combined, leading to the normal PSA and PAP and the decrease of 1CTP significantly.

### Example 2

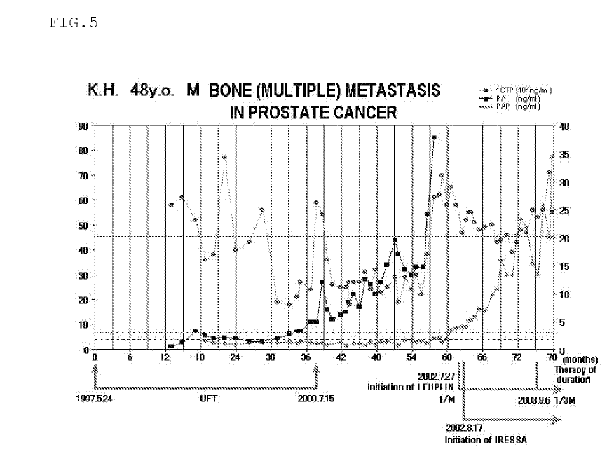

(FIG. 5) 48-year old, a case of prostate cancer and multiple bone metastasis

After starting the treatment with NITC alone, 1CTP was decreased, while PSA and PAP were in a remission state, but from around May in 200X, PSA and 1CTP increased significantly and a generalized bone pain became intense, and even 240 mg/day of morphine could not alleviate the pain. From Jul. 27, 200X, Leuplin (hormone therapy) was started but the pain was not weakened. From Aug. 17, 200X, Iressa 1T (250 mg/day) was initiated. As a result, the generalized bone pain alleviated significantly. Thereafter, though the dosage of Iressa was decreased to 250 mg/alternate day and 250 mg/three days, pains disappeared, and the use of morphine became completely needless. Currently, Iressa is only temporarily and orally administered as a painkiller, when pain appears. As painkiller, Iressa has a stronger effect than morphine.

### INDUSTRIAL APPLICABILITY

As explained above, it was suggested that with the inhibitor of cancer bone metastasis of the present invention, bone metastasis can be inhibited regardless of its site, local or systemic, and the like. Further, it was suggested that with the inhibitor of cancer bone metastasis of the present invention, severe pains caused by osteopathy is alleviated, and further patient's restraint movement and obstacles in daily living resulted from pain can be prevented.

1. A inhibitor of cancer bone metastasis, wherein an inhibition substance of the activation of osteoclast caused by the degradation of a signaling molecule, TRAF6, in the activation of osteoclast, a suppressive substance of the differentiation from osteoclast precursor cells to mature osteoclasts, and/or a bone resorption inhibitor and/or a Cox2 synthesis inhibitor are combined.

2. A inhibitor of cancer bone metastasis, wherein an IL-12 production inducer as an inhibition substance of the activation of osteoclast caused by the degradation of a signaling molecule, TRAF6, in the activation of osteoclast, a tyrosine kinase inhibitor as a suppressive substance of the differentiation from osteoclast precursor cells to mature osteoclasts, and/or a bisphosphonate as a bone resorption inhibitor and/or a Cox2 synthesis inhibitor for inhibiting the stimulation of RANKL/RANK receptor are combined.

3. A inhibitor of cancer bone metastasis, wherein an inhibition substance of the activation of osteoclast caused by the degradation of a signaling molecule, TRAF6, in the activation of osteoclast, a suppressive substance of the differentiation from osteoclast precursor cells to mature osteoclasts, and/or a bone resorption inhibitor and/or a substance enhancing the production of osteoprotegerin are combined.

4. The inhibitor of cancer bone metastasis according to claim 1, wherein the tyrosine kinase inhibitor has a selectively targeting effect to at least one receptor from the followings:
HER2/neu, HER3, HER4, c-kit, PDGFR, bcr-ab1 and EGFR.

5. The inhibitor of cancer bone metastasis according to claim 1, wherein IL-12 production inducer is a substance having a β1,3/1,6 glucan structure.

6. A method for preventing and treating cancer bone metastases by the inhibitor of cancer bone metastasis according to claim 1.

7. The inhibitor of cancer bone metastasis according to claim 2, wherein the tyrosine kinase inhibitor has a selectively targeting effect to at least one receptor from the followings:
HER2/neu, HER3, HER4, c-kit, PDGFR, bcr-ab1 and EGFR.

8. The inhibitor of cancer bone metastasis according to claim 3, wherein the tyrosine kinase inhibitor has a selectively targeting effect to at least one receptor from the followings:
HER2/neu, HER3, HER4, c-kit, PDGFR, bcr-ab1 and EGFR.

9. The inhibitor of cancer bone metastasis according to claim 2, wherein IL-12 production inducer is a substance having a β1,3/1,6 glucan structure.

10. The inhibitor of cancer bone metastasis according to claim 3, wherein IL-12 production inducer is a substance having a β1,3/1,6 glucan structure.

11. The inhibitor of cancer bone metastasis according to claim 4, wherein IL-12 production inducer is a substance having a β1,3/1,6 glucan structure.

12. A method for preventing and treating cancer bone metastases by the inhibitor of cancer bone metastasis according to claim 2.

13. A method for preventing and treating cancer bone metastases by the inhibitor of cancer bone metastasis according to claim 3.

14. A method for preventing and treating cancer bone metastases by the inhibitor of cancer bone metastasis according to claim 4.

15. A method for preventing and treating cancer bone metastases by the inhibitor of cancer bone metastasis according to claim 5.
